(19) United States
(12) Patent Application Publication
(10) Pub. No.: US 2020/0306254 A1
(43) Pub. Date: Oct. 1, 2020

(54) COMBINATION OF ERK1/2 INHIBITOR COMPOUND WITH GEMCITABINE OR WITH GEMCITABINE AND NAB-PACLITAXEL FOR USE IN TREATMENT OF PANCREATIC CANCER

(71) Applicant: Eli Lilly and Company, Indianapolis, IN (US)

(72) Inventors: Shripad BHAGWAT, Carmel, IN (US); Ramon Velasquez TIU, Indianapolis, IN (US); Wenjuan WU, Carmel, IN (US)

(21) Appl. No.: 16/308,887

(22) PCT Filed: Jun. 22, 2017
(86) PCT No.: PCT/US2017/038810

§ 371 Date: Dec. 11, 2018

Related U.S. Application Data
(60) Provisional application No. 62/356,305, filed on Jun. 29, 2016.

(51) Int. Cl. A61K 31/5377; A61K 31/7068; A61K 31/337; A61P 35/00

(52) U.S. Cl. CPC A61K 31/5377; A61P 35/00; A61K 31/337; A61K 31/7068

(57) ABSTRACT

The present invention provides a combination of an ERK1/2 inhibitor compound, 6,6-dimethyl-2-{2-[(1-methyl-1H-pyrazol-5-yl)amino]pyrimidin-4-yl}-5-[2-(morpholin-4-yl) ethyl]-5,6-dihydro-4H-thieno[2,3-c]pyrrol-4-one, or a pharmaceutically acceptable salt thereof with gemcitabine, or a pharmaceutically acceptable salt thereof preferably hydrochloride, or with gemcitabine, or a pharmaceutically acceptable salt thereof preferably hydrochloride, and nab-paclitaxel, and to methods of using the combination to treat certain disorders, such as pancreatic cancer, including pancreatic ductal adenocarcinoma (PDAC).

The present invention relates to a combination of an ERK1/2 inhibitor compound, 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one or a pharmaceutically acceptable salt thereof, (see PCT/U2015/065940) with gemcitabine, or a pharmaceutically acceptable salt thereof, preferably hydrochloride, or with gemcitabine, or a pharmaceutically acceptable salt thereof, preferably hydrochloride, and nab-paclitaxel and to methods of using these combinations to treat certain disorders, such as pancreatic cancer, including pancreatic ductal adenocarcinoma (PDAC).

The RAS/RAF/MEK/ERK signaling pathway (RAS/MAPK pathway) may be activated by growth factors or activating mutations. Mutationally activated KRAS is present in >90% of PDAC and represents the most frequent and the earliest genetic alteration. Dysregulation of the RAS/MAPK pathway may lead to multiple changes in the expression of several genes involved in cell cycle regulation, differentiation, proliferation, survival, migration and angiogenesis. ERK1/2 are key downstream targets in the RAS/MAPK pathway and inhibitors have been developed to target them in cancers driven by alterations in RAS, RAF and MEK1 pathway including PDAC.

Nab-paclitaxel is an antimicrotubule agent and is indicated as first-line treatment, in combination with gemcitabine, for metastatic adenocarcinoma of the pancreas.

Effective therapies for pancreatic cancer, including PDAC, remain elusive. Thus, there exists a need for alternative treatments for pancreatic cancer, such as novel combination therapies.

Certain combinations of ERK inhibitors and antimetabolites have been contemplated in the art. More particularly, WO 2016/025639 discloses the combination of a specific ERK1/2 inhibitor compound, N-(2-((2-((2-methoxy-5-methylpyridin-4-yl)amino)-5-(trifluoromethyl)pyrimidin-4-yl) amino)-5-methylphenyl) acrylamide, with gemcitabine or with gemcitabine and nab-paclitaxel. Pancreatic cancer treatment is among the cancer types disclosed therein. Clinical trial, NCT02608229, provides an ERK inhibitor, BVD-523, in combination with nab-paclitaxel and gemcitabine in patients with metastatic pancreatic cancer.

However, the present invention discloses herein methods of treating pancreatic cancer that provides enhanced and/or unexpected beneficial therapeutic effects from the combined activity 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof, preferably hydrochloride, in pancreatic cancer, including PDAC, as compared to the therapeutic effects provided by either of these agents alone. Additionally, the present invention discloses herein methods of treating pancreatic cancer that provides enhanced and/or unexpected beneficial therapeutic effects from the combined activity 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof, preferably hydrochloride, and nab-paclitaxel, in pancreatic cancer, including PDAC, as compared to the therapeutic effects provided by any of these three agents alone.

Furthermore, the present invention discloses methods of treating pancreatic cancer, including PDAC, as part of a specific treatment regimen that provides enhanced and/or unexpected beneficial therapeutic effects from the combined activity of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof, preferably hydrochloride, in pancreatic cancer patients, including PDAC patients, as compared to the therapeutic effects provided by either of these agents alone. Additionally, the present invention discloses methods of treating pancreatic cancer, including PDAC, as part of a specific treatment regimen that provides enhanced and/or unexpected beneficial therapeutic effects from the combined activity of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, gemcitabine, or a pharmaceutically acceptable salt thereof, preferably hydrochloride, and nab-paclitaxel, in pancreatic cancer patients, including PDAC patients, as compared to the therapeutic effects provided by any of these three agents alone.

Accordingly, the present invention provides a method of treating pancreatic cancer in a patient, comprising administering to the patient an effective amount of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof. Additionally, the present invention provides a method of treating pancreatic cancer in a patient, comprising administering to the patient an effective amount of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, gemcitabine, or a pharmaceutically acceptable salt thereof, and nab-paclitaxel. The present invention also provides a particular embodiment of the method wherein the pancreatic cancer is PDAC. The present invention additionally provides a particular embodiment of the method wherein the pancreatic cancer is pancreatic endocrine tumors.

The present invention also provides a kit for pancreatic cancer comprising an oral agent including an effective component 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and an injection agent including an effective component gemcitabine, or a pharmaceutically acceptable salt thereof. Additionally, the present invention provides a kit for pancreatic cancer comprising an oral agent including an effective component 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, an injection agent including an effective component gemcitabine, or a pharmaceutically acceptable salt thereof, and an injection agent including an effective component nab-paclitaxel. The present invention also provides a particular embodiment of the kit which further comprises one or more pharmaceutically acceptable carriers, diluents, or excipients. The present invention also provides another particular embodiment of the kit wherein the pancreatic cancer is PDAC.

The present invention also provides 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, for use in simultaneous, separate or sequential combination with gemcitabine, or a pharmaceutically acceptable salt thereof, in the treatment of pancreatic cancer. Additionally, the present invention provides 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, for use in simultaneous, separate or sequential combination with gemcitabine, or a pharmaceutically acceptable salt thereof, and nab-paclitaxel, in the treatment of pancreatic cancer.

The present invention also provides gemcitabine, or a pharmaceutically acceptable salt thereof, for use in simultaneous, separate or sequential combination with 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, in the treatment of pancreatic cancer. Additionally, the present invention provides gemcitabine, or a pharmaceutically acceptable salt thereof, for use in simultaneous, separate or sequential combination with 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and nab-paclitaxel in the treatment of pancreatic cancer.

The present invention also provides nab-paclitaxel, for simultaneous, separate or sequential use in combination with 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof, in the treatment of pancreatic cancer.

The present invention also provides a particular embodiment of the invention, wherein nab-paclitaxel is administered at 125 mg/m² IV over 30-40 minutes on day 1, day 8 and day 15 of a 28 day cycle followed immediately by administration of gemcitabine, or a pharmaceutically acceptable salt thereof, preferably hydrochloride, at 1000 mg/m² IV over 30 minutes on day 1, day 8 and day 15 of a 28 day cycle.

The present invention also provides another particular embodiment of the invention, wherein 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, is administered once daily or twice daily and at a dose of 25 mg to 600 mg via oral administration in combination gemcitabine, or a pharmaceutically acceptable salt thereof, preferably hydrochloride, at 1000 mg/m² IV over 30 minutes on day 1, day 8 and day 15 of a 28 day cycle.

The present invention also provides another particular embodiment of the invention, wherein 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, is administered once daily or twice daily and at a dose of 25 mg to 600 mg via oral administration in combination with nab-paclitaxel administered at 125 mg/m² IV over 30-40 minutes on day 1, day 8 and day 15 of a 28 day cycle followed immediately by administration of gemcitabine, or a pharmaceutically acceptable salt thereof, preferably hydrochloride, at 1000) mg/m² IV over 30 minutes on day 1, day 8 and day 15 of a 28 day cycle.

The present invention also provides for the treatment of pancreatic cancer, wherein the pancreatic cancer includes PDAC, wherein PDAC genotypes can include alterations in BRAF, C-MYC (8q), EGFR (7p), KRAS22 (12p), AKT2 (19q), and AIB1 (20q), as well as deletions, including DPC4 SMAD4/MADH4 (18q), CDKN2A (9p), FHIT (3p), and MKK4 (17p), and/or pancreatic endocrine tumors.

As used herein, the term “kit” refers to a package comprising up to three separate agents, wherein a first agent is 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and a second agent is gemcitabine, or a pharmaceutically acceptable salt thereof, preferably hydrochloride, and a third agent is nab-paclitaxel. A “kit” may also include instructions to administer all or a portion of f these agents to a pancreatic cancer patient.

As used herein, the terms “treating,” “to treat,” or “treatment” refers to restraining, slowing, stopping, reducing, or reversing the progression or severity of an existing symptom, disorder, condition, or disease.

As used herein, the term “patient” refers to a mammal, preferably a human.

As used herein, the term “cancer” refers to or describes the physiological condition in patients that is typically characterized by unregulated cell proliferation. Included in this definition are benign and malignant cancers. Examples of cancer as provided in the present invention include pancreatic cancer, including but not limited to particular types of pancreatic cancer such as PDAC, wherein PDAC genotypes can include alterations in BRAF, C-MYC (8q), EGFR (7p), KRAS22 (12p), AKT2 (19q), and AIB1 (20q), as well as deletions, including DPC4/SMAD4/MADH4 (18q), CDKN2A (9p), FHIT (3p), and MKK4 (17p), and/or pancreatic endocrine tumors.

As used herein, the term “primary tumor” or “primary cancer” refer to the original cancer and not a metastatic lesion located in another tissue, organ, or location in the subject's body.

As used herein, the term “effective amount” refers to the amount or dose of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and to the amount or dose of gemcitabine and to the amount or dose of nab-paclitaxel which, upon single or multiple dose administration to the patient, provides an effective response in the patient under diagnosis or treatment. It is also understood that a combination therapy of the present invention is carried out by administering 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, together with gemcitabine, or a pharmaceutically acceptable salt thereof, preferably hydrochloride, and nab-paclitaxel in any manner which provides effective levels of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one or a pharmaceutically acceptable salt thereof, gemcitabine, or a pharmaceutically acceptable salt thereof, preferably hydrochloride, and nab-paclitaxel in the body.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement of or the severity of the disease or disorder; the response of the individual patient; the particular compound administered; the mode of administration; the bioavailabilitv characteristics of the preparation administered; the dose regimen selected; the use of concomitant medication; and other relevant circumstances.

6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one will be administered orally at the particularly frequency and dose determined separately, but with a frequency preferably of once daily or twice daily and at a dose of 25 mg to 2000) mg, more preferably at a dose of 25 mg to 1000 mg and most preferably at a dose of 25 mg to 600 mg. Nab-paclitaxel will be administered at 125 mg/m² IV over 30-40 minutes on day 1, day 8 and day 15 of a 28 day cycle. Gemcitabine will be administered at 1000 mg/m² IV over 30 minutes on day 1, day 8 and day 15 of a 28 day cycle. Gemcitabine will be administered immediately after administration of nab-paclitaxel.

The free base compound, 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, is preferred. It will be understood by the skilled reader that 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one is capable of forming salts. 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one can react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2002); L. D. Bighley, S. M. Berge, D. C. Monkhouse, in “Encyclopedia of Pharmaceutical Technology’. Eds. J. Swarbrick and J. C. Boylan, Vol. 13, Marcel Dekker, Inc., New York, Basel, Hong Kong 1995, pp. 453-499; S. M. Berge, et al., “Pharmaceutical Salts”, Journal of Pharmaceutical Sciences, Vol 66, No. 1, January 1977.

6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof, preferably hydrochloride, and nab-paclitaxel are preferably formulated as pharmaceutical compositions administered by any route which makes each of these compounds bioavailable. The route of administration may be varied in any way, limited by the physical properties of the drugs and the convenience of the patient and the caregiver.

Preferably, 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, is administered orally. Alternatively, 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, is formulated for parenteral administration, such as intravenous or subcutaneous administration. Preferably, gemcitabine is formulated for parenteral administration, such as intravenous or subcutaneous administration. Most preferably, gemcitabine is formulated for intravenous administration. Preferably, nab-paclitaxel is formulated for parenteral administration, such as intravenous administration. Most preferably, gemcitabine is formulated for intravenous administration. Such pharmaceutical compositions and processes for preparing the same are well known in the art. (See, e.g., Remington: The Science and Practice of Pharmacy, L. V. Allen, Editor, 22^{nd} Edition, Pharmaceutical Press, 2012).

As used herein, the phrase “in combination with” refers to either the administration of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof, preferably hydrochloride, either simultaneously or sequentially in any order, such as, for example, at repeated intervals as during a standard course of treatment for a single cycle or more than one cycle, such that one agent can be administered prior to, at the same time, or subsequent to the administration the other agent, or any combination thereof, or to the administration of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, gemcitabine, or a pharmaceutically acceptable salt thereof, preferably hydrochloride, and nab-paclitaxel, either simultaneously or sequentially in any order, such as, for example, at repeated intervals as during a standard course of treatment for a single cycle or more than one cycle, such that one agent can be administered prior to, at the same time, or subsequent to the administration of any of one or both of the other two agents, or any combination thereof.

Gemcitabine is a nucleoside analogue chemotherapeutic agent that exerts its anti-tumor effect by inhibiting deoxyribonucleic acid (DNA) synthesis. The preferred form of gemcitabine is provided as the pharmaceutically acceptable hydrochloride salt of gemcitabine. The compound and methods of making and using this compound including for the treatment of cancer and more specifically for the treatment of leukemias, sarcomas, carcinomas and myelomas are disclosed in U.S. Pat. No. 5,464,826. Alternative names for gemcitabine as the pharmaceutically acceptable hydrochloride salt include Gemzar®, CAS number 122111-03-9, LY 188011 hydrochloride and cytidine, 2′-deoxy-2′,2′-difluoro-, hydrochloride (1:1).

Nanoparticle-albumin bound (nab)-paclitaxel is an albumin-bound form of paclitaxel and has demonstrated enhanced transport across endothelial cell monolayer and great tumor delivery of paclitaxel in preclinical models. The compound is disclosed in U.S. Pat. No. 4,857,653. Alternative names include Abraxane®, CAS number 33069-62-4, (−)-Paclitaxel and benzenepropanoic acid, β-(benzoylamino)-α-hydroxy-, (2aR,4S,4aS,6R,9S, S11S,12S12aR,12bS)-6,12b-bis(acetyloxy)-12-(benzoyloxy)-2a,3,4,4a,5,6,9,10,11,12,12a, 12b-dodecahydro-4,11-dihydroxy-4a,8,13,13-tetramethyl-5-oxo-7,11-methano-1H-cyclodeca[3,4]benz[1,2-b]oxet-9-yl ester, (αR,βS)—.

As used herein, the compound name “6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one” is an inhibitor of extracellular-signal-regulated kinase (ERK) 1 and extracellular-signal-regulated kinase 2 and refers to the compound with the following structure:

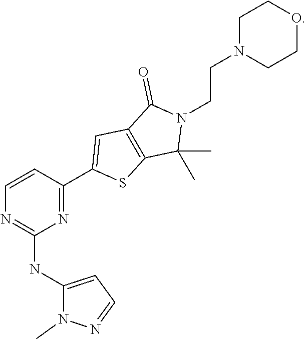 [SMILES: Cn1nccc1Nc1nccc(-c2cc3c(s2)C(C)(C)N(CCN2CCOCC2)C3=O)n1]

This compound can be prepared, for example, using the synthetic steps provided herein below.

As used herein, the following terms have the meanings indicated: “ACN” refers to acetonitrile; “AE” refers to adverse event; “AUC” refers to area under the curve: “DCM” refers to dichloromethane; “DLT” refers to dose limiting toxicity; “DMEM” refers to Dulbecco's Modified Eagle Medium; “DMF” represents N,N-dimethylformamide; “DMSO” refers to dimethyl sulfoxide; “DTT” refers to dithiothreitol; “EDTA” refers to ethylenediaminetetraacetic acid; “EGTA” refers to ethylene glycol tetraacetic acid; “EtOAc” refers to ethyl acetate; “EtOH” refers to ethanol; “FBS” refers to fetal bovine serum; “HBSS” refers to Hank's Balanced Salt Solution; “IC₅₀” refers to half maximal inhibitory concentration; “IV” refers to intravenous; “MS” refers to mass spectroscopy; “MeOH” refers to methanol; “MTD” refers to maximum tolerated dose; “NMR” refers to nuclear magnetic resonance; and “THF” refers to tetrahydrofuran.

### SYNTHETIC STEPS FOR PREPARATION OF COMPOUND A

### Preparation 1

### 6,6-Dimethylthieno[2,3-c]furan-4-one

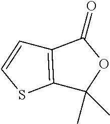

Cool a solution of 3-thiophenecarboxylic acid (250 g, 1.95 mol) in THF (9750 mL) to −70° C. in a 20 L 3-neck flask. To this solution, add n-butyl lithium (2.5 M in hexane, 1872 mL, 4.68 mol) slowly while maintaining the temperature below −55° C. Stir the reaction mixture for one hour at −70° C. Add acetone (187 mL, 2.55 mol) slowly at −70° C. Allow the reaction mixture to warm to 0° C. and stir for three hours at 0° C. To the resulting solution, add 4 M HCl (1500 mL) at 0° C. and allow the reaction mixture to warm to room temperature. Stir the resulting mixture overnight. Filter the reaction mixture through a diatomaceous earth pad and wash the pad with toluene (3×500 mL). Concentrate the filtrate under reduced pressure. Dissolve the resulting crude residue in toluene (3750 mL) and water (250 mL) and add p-toluene sulfonic acid (100.1 g, 0.526 mol) at room temperature. Reflux the reaction mixture for 16 hours at 100° C. Cool the reaction to room temperature and concentrate under reduced pressure at 50° C. Dissolve the resulting residue in water and extract with EtOAc (2×10 L). Wash the organic layer with saturated aqueous sodium bicarbonate and water. Dry the organic layer over anhydrous sodium sulfate, filter and concentrate the filtrate under reduced pressure at 50° C. to provide the title compound 200 g (61%) as brown viscous liquid. MS (m/z): 169 (M+1).

### Preparation 2

### 6,6-Dimethyl-5H-thieno[2,3-c]pyrrol-4-one

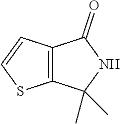

Charge a 5 L autoclave with a solution of 6,6-dimethylthieno[2,3-c]furan-4-one (150 g, 0.891 mol) in ammonium hydroxide (1000 ml). In a closed environment, bring the reaction mixture carefully to a temperature of 200° C. and stir for four hours at 200° C. After four hours, cool the reaction mixture to room temperature and release the ammonia gas. Extract the reaction mixture with DCM (3×750 mL). Wash the organic layer with water (1×750 mL), and dry over anhydrous sodium sulfate, filter and concentrate the filtrate under reduced pressure at 50° C. to give the title compound 100 g (67%). MS (m/z): 168 (M+1).

### Preparation 3

### 2-Bromo-6,6-dimethyl-5H-thieno[2,3-c]pyrrol-4-one

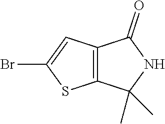

To a 20 L flask containing 6,6-dimethyl-5H-thieno[2,3-c]pyrrol-4-one (835 g, 4.99 mol) add ACN (10000 mL) and cool the solution to 10° C. Add N-bromosuccinimide (444.4 g, 2.49 mol) in four equal portions to the reaction mixture and stir for six hours at 25° C. Concentrate the reaction mixture under reduced pressure and slurry the resulting compound in water and extract with EtOAc (3×4.1 L). Wash the combined organic extracts with water (3×4.1 L) and saturated NaCl (4.1 L), dry over anhydrous sodium sulfate and filter. Store the organic solution for combination with additional batches.

Using the same process as above, prepare two additional batches starting with 650 g and 835 g 6,6-dimethyl-5H-thieno[2,3-c]pyrrol-4-one respectively. Combine the organic solutions from all three runs and concentrate under reduced pressure at 50° C. to yield 2-bromo-6,6-dimethyl-5H-thieno[2,3-c]pyrrol-4-one as brown sticky material. Slurry the resulting product in diethyl ether/hexane (2:1 v/v) and filter to yield the title compound 1542 g (45%). MS (m/z): 246/248 (M+1/M+3).

### Preparation 4

### 4-(2-Bromoethyl)morpholine hydrobromide

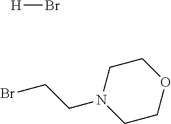

Treat a solution of triphenylphosphine dibromide (124 g, 293 mmol) in DCM (2.44 L) with a solution of 4-morpholineethanol (32 g, 244 mmol) in DCM (60 mL) dropwise over one hour while maintaining the reaction temperature below 25° C. Stir the mixture overnight at room temperature. Conduct an additional reaction as above starting with 4-morpholineethanol (10 g, 76 mmol), scaling the reagents appropriately. Combine the reaction mixtures and collect the solids by vacuum filtration to give the title compound 76.7 g (84%). ¹H NMR (399.8 MHz, DMSO-d₆) δ 4.05 (m, 2H), 3.84 (m, 2H), 3.78 (t, J=7 Hz, 1H), 3.67 (t, J=7 Hz, 2H), 3.56 (m, 2H), 3.26 (m, 2H).

### Preparation 5

### tert-Butyl 2-bromo-6,6-dimethyl-4-oxo-thieno[2,3-c]pyrrole-5-carboxylate

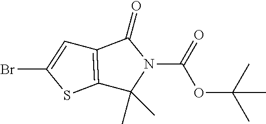 [SMILES: CC(C)(C)OC(=O)N1C(=O)c2cc(Br)sc2C1(C)C]

### Synthetic Method 1:

Treat a solution of 2-bromo-6,6-dimethyl-5H-thieno[2,3-c]pyrrol-4-one (25 g, 102 mmol), 4-dimethylaminopyridine (1.25 g, 10 mmol) and N,N-diisopropylethylamine (24 mL, 138 mmol) in ACN (481 mL) with di-tert-butyldicarbonate (35 g, 162 mmol). Stir the mixture overnight at room temperature. Concentrate the mixture under reduced pressure. Dilute the mixture with hexane, filter the mixture through a silica gel pad and elute the pad with hexane followed by 20% DCM in hexane. Concentrate the filtrate to dryness to give the title compound 36.5 g (93%) as an orange oil. ¹H NMR (399.8 MHz, CDCl₃) δ 7.19 (s, 1H), 1.74 (s, 6H), 1.58 (s, 9H).

### Synthetic Method 2:

Treat a solution of 2-bromo-6,6-dimethyl-5H-thieno[2,3-c]pyrrol-4-one (200 g, 813 mmol), N,N-dimethylpyridin-4-amine (9.93 g, 81 mmol) and di-tert-butyldicarbonate (266 g, 1219 mmol) in ACN (2 L) dropwise with N,N-diisopropylethylamine (213 mL, 1219 mmol). Stir the mixture at room temperature for four hours. Heat the reaction to 30° C. for two hours. Cool the mixture to room temperature and stir overnight. Concentrate the mixture under reduced pressure. Dilute the mixture with EtOAc and wash the resulting organic solution twice with water (300 mL) followed by saturated NaCl (300 mL). Dry the organic solution over anhydrous sodium sulfate, filter and concentrate the filtrate under reduced pressure. Purify the residue on a silica gel pad eluting with a gradient from 0-20% EtOAc in hexane to give the title compound 253 g (90%). MS (m/z): 290/292 (M-isobutene+1/M-isobutene+3). ¹H NMR (399.8 MHz, CDCl₃) δ 7.19 (s, 1H), 1.74 (s, 6H), 1.58 (s, 9H).

### Preparation 6

### tert-Butyl 6,6-dimethyl-4-oxo-2-(4,4,5,5-tetramethyl-1,3,2-dioxaborolan-2-yl)thieno[2,3-c]pyrrole-5-carboxylate

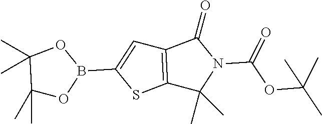 [SMILES: CC(C)(C)OC(=O)N1C(=O)c2cc(B3OC(C)(C)C(C)(C)O3)sc2C1(C)C]

Degas a mixture of tert-butyl 2-bromo-6,6-dimethyl-4-oxo-thieno[2,3-c]pyrrole-5-carboxylate (114 g, 329 mmol), bis(pinacolato)diboron (125 g, 494 mmol) and potassium acetate (97 g, 988 mmol) in 1,4-dioxane (1.6 L) with nitrogen for 10 minutes. Add (1,1′-bis(diphenylphosphino)ferrocene)palladium(II) chloride (5.38 g, 6.6 mmol) and heat the mixture at 90° C. for four hours. Cool the reaction to room temperature and filter through a CELITE® pad. Concentrate the filtrate and then treat the residue with 10% EtOAc in hexane. Collect the precipitate by vacuum filtration to give the title compound 65.8 g (40%). MS (m/z): 338 (M-isobutene+1).

### Preparation 7

### tert-Butyl 2-(2-chloropyrimidin-4-yl)-6,6-dimethyl-4-oxo-thieno[2,3-c]pyrrole-5-carboxylate

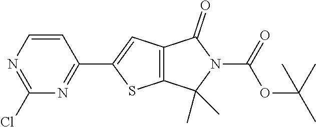 [SMILES: CC(C)(C)OC(=O)N1C(=O)c2cc(-c3ccnc(Cl)n3)sc2C1(C)C]

### Synthetic Method 1:

Degas a mixture of tert-butyl 2-bromo-6,6-dimethyl-4-oxo-thieno[2,3-c]pyrrole-5-carboxylate (36 g, 104 mmol), bis(pinacolato)diboron (59.8 g, 235 mmol) and potassium acetate (33.2 g, 338 mmol) in 1,4-dioxane (520 mL) with nitrogen for 10 minutes. Add (1,1′-bis(diphenylphosphino)ferrocene)palladium(II) chloride (4.45 g, 5.5 mmol) and heat the mixture to 90° C. Heat the mixture at 90° C. for two hours. Cool the reaction to room temperature and stir for three hours. Add 2,4-dichloropyrimidine (22 g, 145 mmol) followed by a solution of potassium carbonate (20.4 g, 147 mmol) in water (83 mL). Degas the resulting mixture with nitrogen for 10 minutes. Add tetrakis(triphenylphosphine)palladium (1.59 g, 1.38 mmol) and heat the mixture to 90° C. for two hours. Cool the mixture to room temperature and filter through a pad of CELITE®. Wash the filtrate with three portions water and one portion of saturated NaCl. Concentrate the organics under reduced pressure. Purify the residue by silica gel column chromatography eluting with a gradient from 0-25% EtOAc in DCM to give the title compound 11 g (59%). MS (m/z): 324 (M-isobutene+1).

### Synthetic Method 2

Degas a mixture of tert-butyl 6,6-dimethyl-4-oxo-2-(4,4,5,5-tetramethyl-1,3,2-dioxaborolan-2-yl)thieno[2,3-c]pyrrole-5-carboxylate (11.7 g, 30 mmol), 2,4-dichloropyrimidine (13 g, 89 mmol), potassium carbonate (20.4 g, 147 mmol), and water (50 mL) in 1,4-dioxane (100 mL) with nitrogen for 10 minutes. Add tetrakis(triphenylphosphine)palladium (2.58 g, 2.2 mmol) and heat the mixture to 87° C. for 1.5 hours. Cool the mixture to room temperature. Dilute the mixture with EtOAc (1 L) and wash the resulting solution with water and saturated NaCl. Dry the organic solution over anhydrous sodium sulfate, filter and concentrate the filtrate under reduced pressure. Treat the residue with 30% EtOAc in hexane (200 mL) and collect the resulting precipitate by vacuum filtration to give the title compound 7.6 g (67%). MS (m/z): 324 (M-isobutene+1).

### Preparation 8

### 2-(2-Chloropyrimidin-4-yl)-6,6-dimethyl-5H-thieno[2,3-c]pyrrol-4-one

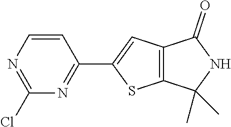 [SMILES: CC1(C)NC(=O)c2cc(-c3ccnc(Cl)n3)sc21]

Stir a mixture of tert-butyl 2-(2-chloropyrimidin-4-yl)-6,6-dimethyl-4-oxo-thieno[2,3-c]pyrrole-5-carboxylate (6.36 g, 16.7 mmol) and trifluoroacetic acid (25 mL) in DCM (25 mL) at room temperature for two hours. Concentrate the mixture under reduced pressure and dilute the residue with DCM. Partition the mixture with saturated aqueous sodium bicarbonate solution and collect the solids from the biphasic emulsion. Wash the solids with ether and dry under vacuum at 50° C. overnight to give the title compound 4.65 g (99%). MS (m/z): 280 (M+1).

### Preparation 9

### 2-(2-Chloropyrimidin-4-yl)-6,6-dimethyl-5H-thieno[2,3-c]pyrrol-4-one hydrochloride

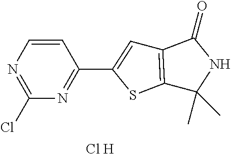 [SMILES: CC1(C)NC(=O)c2cc(-c3ccnc(Cl)n3)sc21 ; "Cl H"]

Heat a solution of tert-butyl 2-(2-chloropyrimidin-4-yl)-6,6-dimethyl-4-oxo-thieno[2,3-c]pyrrole-5-carboxylate (66.7 g, 176 mmol) and hydrogen chloride (4 M in 1,4-dioxane, 263 mL, 1054 mmol) in 1,4-dioxane (585 mL) at 30° C. for five hours. Remove the heating element and stir the mixture at room temperature overnight. Slowly add hexane (800 mL) to the reaction mixture. Stir the resulting slurry for 10 minutes and collect the solids by vacuum filtration. Dry the solid under vacuum to give the title compound 56 g (100%). MS (m/z): 280 (M+1).

### Preparation 10

### 2-Bromo-6,6-dimethyl-5-(2-morpholinoethyl)thieno[2,3-c]pyrrol-4-one

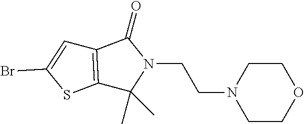 [SMILES: CC1(C)c2sc(Br)cc2C(=O)N1CCN1CCOCC1]

Add sodium hydroxide (160 g, 4 mol) to water (250 mL) and stir the mixture until a clear solution is produced. Add 1,4-dioxane (2 L) followed by 2-bromo-6,6-dimethyl-5H-thieno[2,3-c]pyrrol-4-one (215 g, 874 mmol), tetrabutylammonium iodide (300 g, 812 mmol) and 4-(2-chloroethyl)morpholine hydrochloride (300 g, 1564 mmol). Heat the mixture at 80° C. for one hour. Cool the reaction mixture to room temperature. Dilute the reaction with water (2 L) and extract the mixture with EtOAc (3×2 L). Dry the combined organic extracts over anhydrous sodium sulfate, filter and concentrate the filtrate under reduced pressure. Add DCM (2 L) and hexane (2 L) and wash the resulting organic solution with saturated NaCl (2×1 L). Concentrate the organic solution under reduced pressure to a minimum volume. Filter off the solids to give the title compound 180 g (57%). MS (m/z): 359/361 (M+1/M+3).

### Preparation 11

### 2-(2-Chloropyrimidin-4-yl)-6,6-dimethyl-5-(2-morpholinoethyl)thieno[2,3-c]pyrrol-4-one

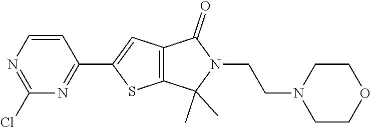 [SMILES: CC1(C)c2sc(-c3ccnc(Cl)n3)cc2C(=O)N1CCN1CCOCC1]

### Synthetic Method 1:

Degas a mixture of 2-bromo-6,6-dimethyl-5-(2-morpholinoethyl)thieno[2,3-c]pyrrol-4-one (200 g, 557 mmol), bis(pinacolato)diboron (200 g, 788 mmol) and potassium acetate (200 g, 2038 mmol) in 1,4-dioxane (1 L) with nitrogen for 15 minutes. Add (1,1′-bis(diphenylphosphino)ferrocene)palladium(II) chloride (20 g, 27 mmol) and heat the mixture to 90° C. Heat the mixture at 90° C. for one hour. Cool the reaction to 50° C. and add potassium carbonate (250 g, 1809 mmol), 2,4-dichloropyrimidine (230 g, 1543 mmol) and water (300 mL). Heat the mixture at 90° C. for one hour. Cool the mixture to 35° C. and add water (700 mL). Extract the reaction mixture with DCM (2 L). The aqueous solution was back extracted with DCM (500 mL). Dry the combined organic solutions over anhydrous magnesium sulfate, filter and concentrate the filtrate under reduced pressure. Dilute the residue with 10% EtOAc in hexane (2 L) and stir for one hour. Decant the mother liquor and rinse the solids with hexane (500 mL). Dissolve the solids in DCM (300 mL) and slowly add hexanes (2 L). Collect the resulting solids by vacuum filtration and dry to give the title compound 150 g (65%). MS (m/z): 393 (M+1).

### Synthetic Method 2:

Cool a mixture of 2-(2-chloropyrimidin-4-yl)-6,6-dimethyl-5H-thieno[2,3-c]pyrrol-4-one hydrochloride (10 g, 32 mmol) and tetrabutylammonium iodide (1.17 g, 3.16 mmol) in N-methylpyrrolidone (211 mL) to 0° C. using an ice water bath. Add sodium hydride (60 wt % in mineral oil, 5.06 g, 126.5 mmol) in portions. Stir the mixture at 0° C. for 10 minutes and then add 4-(2-bromoethyl)morpholine hydrobromide (13.9 g, 50.6 mmol). Remove the ice bath and stir the mixture for four hours. Quench the reaction mixture with saturated aqueous ammonium chloride and dilute the mixture with water (1 L). Extract the mixture with isopropyl acetate (4×700 mL). Dry the combined organic extracts over anhydrous sodium sulfate, filter and concentrate the filtrate under reduced pressure. Add 20% EtOAc in hexane and stir the mixture for one hour. Collect the solid by vacuum filtration and dry to give the title compound 8.6 g (69%). MS (m/z): 393 (M+1).

### Synthetic Method 3:

Treat a solution of 2-(2-chloropyrimidin-4-yl)-6,6-dimethyl-5H-thieno[2,3-c]pyrrol-4-one (500 mg, 1.4 mmol) in DMF (14 mL) with sodium hydride (60 wt % in mineral oil, 129 mg, 3.2 mmol). Stir the mixture for 10 minutes and then add 4-(2-bromoethyl)morpholine hydrochloride (412 mg, 1.8 mmol). Stir the reaction mixture at room temperature overnight. Cool the mixture to 0° C. and add 4-(2-bromoethyl)morpholine hydrochloride (165 mg, 0.7 mmol) followed by sodium hydride (60 wt % in mineral oil, 14 mg, 0.4 mmol). Remove the ice bath and stir the mixture at room temperature overnight. Add sodium hydride (60 wt % in mineral oil, 14 mg, 0.4 mmol) and stir the resulting mixture at room temperature for five hours. Dilute the mixture with water and extract with EtOAc. Wash the organic extracts with 5% aqueous lithium chloride. Concentrate the organic solution under reduced pressure. Purify the residue by silica gel column chromatography eluting with a gradient from 0-10% MeOH in DCM to give the title compound 524 mg (93%). MS (m/z): 393 (M+1).

### Compound A

### 6,6-Dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one

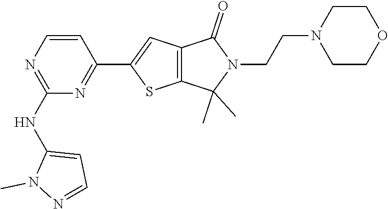 [SMILES: Cn1nccc1Nc1nccc(-c2cc3c(s2)C(C)(C)N(CCN2CCOCC2)C3=O)n1]

### Synthetic Method 1:

Slowly add 2-methylpyrazol-3-amine (75 g, 772 mmol) to a suspension of sodium hydride (60 wt/in mineral oil, 30 g, 750 mmol) in N-methylpyrrolidone (500 mL). Stir the resulting mixture for 90 minutes. Add a solution of 2-(2-chloropyrimidin-4-yl)-6,6-dimethyl-5-(2-morpholinoethyl)thieno[2,3-c]pyrrol-4-one (145 g, 369 mmol) in N-methylpyrrolidone (200 mL). Cool the exothermic reaction to room temperature and pour the reaction into water (3 L). Adjust the pH to ˜3 with concentrated hydrochloric acid (200 mL). Extract the mixture with DCM (4×2 L). Neutralize the aqueous layer using 5 M sodium hydroxide. Extract this aqueous solution with DCM (2×2 L). Combine these organic extracts and wash with water (2 L). Dry the organics over anhydrous sodium sulfate, filter and concentrate the filtrate under reduced pressure. Purify the residue on a silica gel plug (2 kg) eluting successively with DCM (2 L), 2.5% EtOH in DCM (2 L), 5% EtOH in DCM (2 L), 7.5% EtOH in DCM (2 L) and finally 10% EtOH in DCM (10 L). Concentrate the appropriate fractions under reduced pressure. Add EtOAc (1 L) and concentrate under reduced pressure. Add EtOAc (1 L) and concentrate under reduced pressure. Add EtOAc (500 mL) and hexane (500 mL). Collect the solid by vacuum filtration and wash the solid with hexane (500 mL). Dry the solid under vacuum at 50° C. to give the title compound 65.7 g (39%). MS (m/z): 454 (M+1).

### Synthetic Method 2:

Degas a mixture of 2-(2-chloropyrimidin-4-yl)-6,6-dimethyl-5-(2-morpholinoethyl)thieno[2,3-c]pyrrol-4-one (20.8 g, 52.9 mmol), 2-methylpyrazol-3-amine (5.7 g, 58.2 mmol), cesium carbonate (37.9 g, 116.5 mmol), 4,5-bis(diphenylphosphino)-9,9-dimethylxanthene (2.6 g, 4.5 mmol) and 1,4-dioxane (529 mL) with nitrogen for 10 minutes. Add tris(dibenzylideneacetone)dipalladium(0) (2.4 g, 2.6 mmol) and heat the mixture to 85° C. for four hours. Cool the mixture to room temperature and filter the mixture through filter paper. Concentrate the filtrate under reduced pressure. Repeat the reaction starting with 8 g of 2-(2-chloropyrimidin-4-yl)-6,6-dimethyl-5-(2-morpholinoethyl)thieno[2,3-c]pyrrol-4-one and combine the two residues. Purify the residue by silica gel column chromatography (330 g) eluting with a gradient from 5-25% MeOH in (10% EtOAc in DCM). Pool the fractions and concentrate under reduced pressure. Re-purify the residue by silica gel column chromatography (330 g) eluting with a gradient from 5-25% MeOH in 10% EtOAc in DCM. Pool the fractions and concentrate under reduced pressure. Dissolve the residue in DCM (400 mL) and then add acetone (1 L). Slowly concentrate the mixture under reduced pressure to approximately 700 mL. Collect the solid by vacuum filtration to give the title compound 14.8 g (48%). MS (m/z): 454 (M+1).

### Synthetic Method 3:

Degas a mixture of 2-(2-chloropyrimidin-4-yl)-6,6-dimethyl-5-(2-morpholinoethyl)thieno[2,3-c]pyrrol-4-one (250 mg, 0.64 mmol), 2-methylpyrazol-3-amine (124 mg, 1.3 mmol), cesium carbonate (622 mg, 1.9 mmol), 4,5-bis(diphenylphosphino)-9,9-dimethylxanthene (55 mg, 0.095 mmol) and 1,4-dioxane (6.4 mL) with nitrogen for 15 minutes. Add palladium(II)acetate (14.3 mg, 0.0636 mmol) and heat the mixture at 90° C. overnight. Cool the mixture to room temperature and filter the mixture through filter paper. Wash the solids with 10% MeOH in DCM. Concentrate the filtrate under reduced pressure. Repeat the reaction and combine the two residues. Purify the residue by HPLC on a C18 column (30×75 mm, 5 um, xbridge ODB) eluting with a 85 mL/minute gradient from 9-28% ACN in 10 mM ammonium carbonate (pH 10) in water. Pool the fractions and concentrate under reduced pressure to remove the ACN. Lyophilize the aqueous solution to give the title compound 100 mg (18%). MS (m/z): 454 (M+1).

### Biological Assays for Compound A

### ERK1 Kinase Assay

The purpose of this assay is to measure the ability of a compound to inhibit ERK1 kinase activity. Perform the ERK1 kinase assay in vitro using a TR-FRET assay. Start reactions (12.5 μL) by adding 5 μL of ERK1 enzyme (Invitrogen, # PR5254B, final concentration 100 ng/mL) plus substrate GFP-ATF2 (Invitrogen, # PV4445, final concentration 0.2 μM), 5 μL of ATP solution (Invitrogen, # PV3227, final concentration M) prepared in kinase buffer (50 mM Hepes pH 7.4, 5 mM MgCl₂, 0.1 mM EGTA, 0.01% Triton X-100, 1 mM DTT) and 2.5 μL of testing compounds in DMSO solution (final 4%, v/v) in a 384-well PROXIPLATE™ (Perkin Elmer, # GRN6260). Incubate the reaction mixture at room temperature for 60 minutes. Stop the reaction by addition of 12.5 μL of stop buffer (10 mM EDTA, 2 nM Tb-anti-pATF2 (pThr71) antibody, Invitrogen, # PV4448) in TR-FRET dilution buffer (Invitrogen, # PV3574). Incubate the plates at room temperature for an additional 60 minutes and read on an ENVISION® (PerkinElmer) plate reader at the excitation wavelength 340 nm. Calculate the TR-FRET ratio by dividing the GFP acceptor emission signal (at 520 nm) by the Tb donor emission signal (at 495 nm). Calculate percent inhibition using compound treated wells relative to on-plate Max (DMSO control) and Min (No enzyme added) control wells TR-FRET ratio data {% inhibition=100−[(test compound−median Min)/(median Max−median Min)×100]}. Test all compounds at 10 concentrations (20 μM to 0.001 μM) using a 1:3 dilution scheme. Derive Abs_IC₅₀ values by fitting percent inhibition and ten-point concentration data to a 4-parameter nonlinear logistic equation (equation 205) using ACTIVITYBASE® 7.3 (ID Business Solutions Limited).

Compound A is tested in this assay substantially as described above. The results of this assay demonstrates that Compound A inhibits ERK1 kinase activity and has an IC₅₀ value of 4.86 nM (±0.20, n=7).

### ERK2 Kinase Assay

The purpose of this assay is to measure the ability of a compound to inhibit ERK2 kinase activity. Perform the ERK2 kinase assay in vitro using a TR-FRET assay. Start all reactions (12.5 μL) by adding 5 μL of ERK2 enzyme (Invitrogen, # PV3595B, final conc 50 ng/mL) plus substrate GFP-ATF2 (Invitrogen, # PV4445, final conc 0.2 μM), 5 μL of ATP solution (Invitrogen, # PV3227, final conc 10 μM) prepared in kinase buffer (50 mM Hepes pH 7.4, 5 mM MgCl₂, 0.1 mM EGTA, 0.01% Triton X-100, 1 mM DTT) and 2.5 μL of testing compounds in DMSO solution (final 4%, v/v) in a 384-well PROXIPLATE™ (Perkin Elmer, # GRN6260). Incubate reactions at room temperature for 60 minutes. Stop reactions by addition of 12.5 μL of stop buffer (10 mM EDTA, 2 nM Tb-anti-pATF2 (pThr71) antibody, Invitrogen, # PV4448) in TR-FRET dilution buffer (Invitrogen, # PV3574). Incubate the plates at room temperature for an additional 60 minutes and read ON ENVISION® (PerkinElmer) plate reader at the excitation wavelength of 340 nm. Calculate a TR-FRET ratio by dividing the GFP acceptor emission signal (at 520 nm) by the Tb donor emission signal (at 495 nm). Calculate percent inhibition using compound wells relative to on-plate Max (DMSO control) and Min (No enzyme added) control wells TR-FRET ratio data {% inhibition=100−[(test compound−median Min)/(median Max−median Min)×100]}. Test all compounds at 10 concentrations (20 μM to 0.001 μM) using a 1:3 dilution scheme. Derive Abs_IC50 values by fitting percent inhibition and ten-point concentration data to a 4-parameter nonlinear logistic equation (equation 205) using ACTIVITYBASE 7.3 (ID Business Solutions Limited).

Compound A is tested in this assay substantially as described above. The results of this assay demonstrate that Compound A inhibits ERK2 kinase activity and has an IC₅₀ value of 5.24 nM (+0.24, n=7).

### ERK1/2 Cell Mechanistic Assay (pRSK1 Alphascreen Assay)

The purpose of this assay is to measure the ability of a compound to inhibit ERK signaling in cancer cells in vitro. Carry out the pRSK1 Alphascreen assay using the HCT116 colorectal cancer cell line (ATCC, # CCL-247). Routinely culture HCT116 cells in Dulbecco's Modified Eagle's Medium (DMEM) (Hyclone, # SH30022) growth medium containing 5%0/Fetal Bovine Serum (FBS) (Gibco, #16000-044) in T-150 flasks and incubate in a 5% CO₂ incubator at 37° C. Harvest cells when they become confluent and freeze in freezing medium at 1×10e⁷ cells/mL as “assay ready frozen cells” and store in liquid nitrogen. To run the assay, plate 40,000 HCT116 cells/well in a 96-well tissue culture plate and incubate at 37° C. in a 5% CO₂ incubator overnight. Test compounds at 10 concentrations starting at a 20 μM top concentration and utilize a 1:3 dilution scheme (20 μM to 0.001 μM) with a final DMSO concentration of 0.5% (v/v). Add compounds in 20 μL serum free growth medium and incubate at 37° C. for two hours. Remove growth medium and add 50 μL of 1× lysis buffer [Cell Signaling Technology, #9803] containing 1× holt protease and phosphate inhibitor cocktail [Thermo, #78441] to each well and incubate at room temperature for 10 minutes on a shaker. Transfer 4 μL of cell lysate from each well to respective wells in a 384 well assay plate [Perkin Elmer, #6006280] and add 5 μL of reaction mix [2000 parts 1× assay buffer (Perkin Elmer, # A1000), 1 part biotin-RSK1 antibody (Santa Cruz, # sc-231-B-G), 4 parts pRSK1 antibody (Abcam, # ab32413), 35 parts acceptor beads (Perkin Elmer, #6760617R)]. Seal the plate with foil plate seal (Beckman Coulter, #538619) and incubate at room temperature for two hours. Add 2 JAL of donor beads [20 parts 1× assay buffer, 1 part donor beads] to each well and seal the plate with clear plate seal (Applied Biosystems, #4311971) and incubate at room temperature in the dark for two hours. Measure the fluorescence intensity in each well by reading the plates in ENVISION® (PerkinElmer) plate reader. Derive the Rel IC₅₀ values by fitting percent pRSK1 inhibition [% inhibition=100−[(test compound−median Min)/(median Max−median Min)×100] and ten-point concentration data to a 4-parameter nonlinear logistic equation (Abase equation 205) using ACTIVITYBASE® 7.3 (ID Business Solutions Limited).

Compound A is tested in this assay substantially as described above. The results of this assay demonstrate that Compound A inhibits ERK substrate (RSK) phosphorylation in tumor cells and has an IC₅₀ value of 0.429 μM (+0.173, n=8).

### Xenograft Tumor Model

The purpose of this assay is to measure reduction in tumor volume in response to test compound administration. Expand human pancreatic cancer cells MIA PaCa2 (ATCC, # CRL1420) in culture, harvest and inject 5×10e⁶ cells in 200 μL of 1:1 HBSS+matrigel solution subcutaneously on the rear right flank of female athymic nude mice (22-25 g, Harlan Laboratories). Measure tumor growth and body weight twice per week beginning the seventh day after the implantation. When tumor sizes reach 200-400 mm³, randomize animals and group into groups of eight to ten animals. Prepare test compound in an appropriate vehicle (vehicle: 10/% HEC/0.25% TWEEN® 80/0.05% Antifoam) and administer by oral gavage daily for 14 days. Tumor response is determined by tumor volume measurement performed twice a week during the course of treatment. Body weight is taken as a general measure of toxicity.

Compound A is tested in this assay substantially as described above. Compound A is found to have delta T/C % values as provided in Table 1 below. These results indicate that Compound A demonstrates significant anti-tumor activity in a human pancreatic cancer xenograft model (MIA PaCa2).

TABLE 1
Efficacy of Example 1 in human pancreatic cancer xenograft model

| | | | | Delta T/C % or |
|---|---|---|---|---|
| Tumor Model | Dose (mg/kg) | Schedule | p-value | Regression % |
| MIA PaCa-2 | 12.5 | QD | 0.003* | 32 |
| MIA PaCa2 | 25 | QD | <0.001* | 2 |
| MIA PaCa2 | 50 | QD | <0.001* | −22 |
| MIA PaCa2 | 100 | QD | <0.001* | −66 |

Analysis for tumor volume is based on Log 10 and SpatialPower covariance structure.
*significant (p < 0.05)
NA: Not applicable
Della T/C % is calculated when the endpoint tumor volume in a treated group is at or above baseline tumor volume. The formula is 100*(T − T₀)/(C− C ₀), where T and C are mean endpoint tumor volumes in the treated or control group, respectively. T₀ and C₀ are mean baseline tumor volumes in those groups.

Regression % is calculated when the endpoint volume is below baseline. The formula is 100*(T−T₀)/T₀. Where T₀ is the mean baseline tumor volume for the treated group.
Grand mean of all groups from baseline (randomization) at day 20 used to compute % change of T/C.

### Combination of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one (Compound A) with Gemcitabine in Xenograft Model of Pancreatic Cancer

In the current study, in vivo combination efficacy of ERK inhibitor Compound A with gemcitabine (prior standard of care for pancreas cancer patient) is evaluated on tumor growth in KRAS mutant (G12V) Capan-2 pancreatic cancer xenograft model. The combination results in significant (p<0.001) tumor growth regression (14%). The combination effect is additive and also is tolerated in mice.

### Cell Culture

Human pancreatic cancer cell line Capan-2 (ATCC cat # HTB-80) are cultured in DMEM supplemented with 10% fetal bovine serum, sodium pyruvate, nonessential amino acids, L-glutamine, and penicillin-streptomycin (Invitrogen, Carlsbad, Calif.). All cultures are maintained in a humidified incubator at 37° C. under 5% CO₂/95% air free off mycoplasma and pathogenic murine viruses. The cells are used for experiments at passages <7 after recovery from frozen stocks.

### Animals

Female CB-17 SCID nude mice are ordered from Charles River Laboratories International, Inc. All animals are acclimated for one week before the use and are housed and maintained in specific pathogen-free conditions in accordance with the guidelines of the American Association for Laboratory Animal Care and all current regulations and standards of the U.S. Departments of Agriculture and of Health and Human Services and the NIH. The experiment protocol is approved by the Eli Lilly and Company Animal Care and Use Committee.

Xenograft Models and Therapeutic Treatment with Compounds In Vivo

Logarithmically growing Capan-2 cells (5×10⁶/200 μL, single-cell suspensions of over 95% viability in Hank's medium mixed with equal volume of Matrigel (Becton Dickinson & Co., San Jose, Calif.)) are subcutaneously injected into the flank of the mice. When the average tumor volume reaches about 200 to 300 mm³ following tumor cell implantation, the animals are randomized into different groups (n=5). The animals are treated with vehicle control (1% HEC/0.25% TWEEN® 80/0.05% Antifoam, daily, orally), Compound A (50 mg/kg, QD, PO), gemcitabine (100 mg/kg, once weekly, intraperitoneal), and the combination for 4 weeks. Compound A was formulated in 1% HEC/0.25% TWEEN® 80/0.05% Anti foam and gemcitabine is formulated in phosphate buffered saline (lx). Tumor volume and body weight are measured twice weekly. Tumor volume is estimated by using the formula: v=l×w2×0.536 where l=larger of measured diameter and w=smaller of perpendicular diameter.

### Statistical Analysis

The statistical analysis of the tumor volume data begins with a data transformation to a log scale to equalize variance across time and treatment groups. The log volume data are analyzed with a two-way repeated measures analysis of variance by time and treatment using the MIXED procedures in SAS software (Version 9.3). The correlation model for the repeated measures is Spatial Power. Treated groups are compared to the control group at each time point. The MIXED procedure is also used separately for each treatment group to calculate adjusted means and standard errors at each time point. Both analyses account for the autocorrelation within each animal and the loss of data that occurs when animals with large tumors are removed from the study early. The adjusted means and standard errors (s.e.) are plotted for each treatment group versus time. Analysis for tumor volume is based on log₁₀ and spatial power covariance structure. P value is based on the comparison between two specific groups.

### Combination Analysis Method (Bliss Independence for IVEF Studies)

First, the usual repeated measures model is fit to log volume versus group and time. Then contrast statements are used to test for an interaction effect at each time point using the 2 specific treatments that are combined. This is equivalent to the Bliss Independence method and assumes that tumor volumes can, in theory, reach zero, i.e., complete regression. The expected additive response (EAR) for the combination is calculated on the tumor volume scale as: response (EAR) EAR volume=V1*V2/V0, where V0, V1, and V2 are the estimated mean tumor volumes for the vehicle control, treatment 1 alone, and treatment 2 alone, respectively. If the interaction test is significant, the combination effect is declared statistically more than additive or less than additive depending on the observed combination mean volume being less than or more than the EAR volume, respectively. Otherwise, the statistical conclusion is additive. In addition, a biologically relevant range of additivity can be defined as X % above and below the EAR volume. Typically, X would be 25 to 40%. Then a biological conclusion can be made for the combination as more than additive, additive, or less than additive if the observed combination mean volume is below, in, or above the interval of additivity.

There may be situations were stasis is the best expected response. In those situations, the Bliss method can be applied directly to the % delta T/C values to obtain an EAR percent response: EAR % delta T/C=Y1*Y2/100, where Y1 and Y2 are the percent delta T/C values for the single-agent treatments. Currently, there is no statistical test to compare the observed % delta T/C in the combination group versus the EAR, but the biological criterion described above can be applied.

Treatment with Compound A or gemcitabine alone result in a partial inhibition of tumor growth when compared with the vehicle control group; and the combination shows the superior inhibition of tumor growth (p<0.001) when compared with each single agent alone. As shown in Table 2 and Table 3, Compound A (50 mg/kg) and gemcitabine (100 mg/kg) as single agent lead to 40% (p=0.054) and 20% (p=0.005) Delta T/C, respectively; and the combination results in additive effect with 14% tumor regression on day 34 following tumor implantation (Table 3). The combination is tolerated in the animals without significant body weight loss.

TABLE 2
Combination Efficacy of Compound A with Gemcitabine on Tumor
Growth in Capan-2 Pancreas Cancer Xenograft Tumor Model

| | | Differ- | | |
|---|---|---|---|---|
| Treatment 1 | Treatment 2 | ence^{b} | SE | p-value |
| Vehicle, | Compound A, 50 | 0.397 | 0.0844 | <0.001* |
| QD x 28, PO | mpk, QD x 28, PO | | | |
| Vehicle, | Gemcitabine, 100 | 0.633 | 0.0796 | <0.001* |
| QD x 28, PO | mpk, Q7D x 4, IP | | | |
| Vehicle, | Gemcitabine, 100 | 0.926 | 0.0796 | <0.001* |
| QD x 28, PO | mpk, Q7D x 4, IP/ | | | |
| | Compound A, 50 | | | |
| | mpk, QD x 28, PO | | | |
| Compound A, 50 | Gemcitabine, 100 | 0.529 | 0.0844 | <0.001* |
| mpk, QD x 28, PO | mpk, Q7D x 4, IP/ | | | |
| | Compound A, 50 | | | |
| | mpk, QD x 28, PO | | | |
| Gemcitabine, 100 | Gemcitabine, 100 | 0.293 | 0.0796 | <0.001* |
| mpk, Q7D x 4, IP | mpk, Q7D x 4, IP/ | | | |
| | Compound A, 50 | | | |
| | mpk, QD x 28, PO | | | |

^{b}Difference = Treatment 1 − Treatment 2;
*p-value: significant (p < 0.05)
SE—Standard error

TABLE 3
Combination Efficacy of Compound A with Gemcitabine
on Tumor Growth (Day 34) in Capan-2 Pancreas
Cancer Xenograft Tumor Model

| | Delta T/C % | | Combination | |
|---|---|---|---|---|
| Treatment | or % Regression | p-value | Effect | Bodyweight |
| Vehicle | NA | NA | | |
| Compound A | 40 | 0.054 | | |
| Gemcitabine | 24 | 0.005* | | |
| Compound A/ | −14 | <0.001* | Additive | No |
| Gemcitabine | | | | significant |
| (Combination) | | | | change |

Analysis for tumor volume is based on Log₁₀ and Spatial Power covariance structure.
*p-value: significant (p < 0.05);
NA: Not applicable
Delta T/C % is calculated when the endpoint tumor volume in a treated group is at or above baseline tumor volume; and regression % is calculated for tumor volume below the baseline. The formula is 100*(T − T₀)/(C − C₀), where T and C are mean endpoint tumor volumes in the treated or control group, respectively. T₀ and C₀ are mean baseline tumor volumes in those groups.

### A Phase 1 Trial of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one (Compound A) in Combination with Gemcitabine and Nab-Paclitaxel

### Study Design

The purpose of the study, in part, is to assess the safety and tolerability of 6,6-dimethyl-2-({2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one (Compound A) in combination with nab-paclitaxel/gemcitabine.

Study Objectives and Endpoints: Combination with Gemcitabine and Nab-Paclitaxel

To assess safety and tolerability of Compound A administered in combination with nab-paclitaxel plus gemcitabine in metastatic PDAC.

### Treatment Plan

Compound A will be administered orally at the particularly frequency and dose determined separately, but with a frequency preferably of once daily or twice daily and at a dose of 25 mg to 2000 mg, more preferably at a dose of 25 mg to 1000 mg and most preferably at a dose of 25 mg to 600 mg. Nab-paclitaxel will be administered per label, 125 mg/m² IV over 30-40 minutes on day 1, day 8 and day 15 of a 28 day cycle. Gemcitabine will be administered per label, 1000 mg/m² IV over 30 minutes on day 1, day 8 and day 15 of a 28 day cycle. Gemcitabine will be administered immediately following administration of nab-paclitaxel.

### Dose Escalation

Dose escalation will be driven by safety using the 3+3 method. Each new dose level will have a minimum of 3 patients enrolled to it. If 1 patient, at any dose level, experiences a DLT within the first cycle of Compound A, then up to 3 additional patients will be enrolled at that dose level. If a DLT is observed in 2 or more patients at any dose level, dose escalation will cease and either the previous dose level will be declared the MTD or, following discussions between the sponsor and investigators additional patients may be treated at intermediate doses between the previous and current dose levels. The MTD is defined as the highest tested dose that has <33% probability of causing a DLT during Cycle 1 in a cohort of at least 6 patients. Determination of the recommended dose will take into account toxicities beyond Cycle 1, PK, and dose modifications of combination therapy. Nab-paclitaxel and gemcitabine will be administered per label, immediately following the dose of Compound A.

During combination treatment, when a study drug is delayed, if possible and appropriate, patients should resume study treatment within 1 treatment cycle, with every effort made to start on the first day of the next dosing schedule, with all study drugs administered (as appropriate). All dose modifications should be documented, including the approach taken and a clear rationale for the need for modification. For the purposes of dose modification, the investigator must first assess if the toxicity is considered at least possibly due to one of the study drugs, and must then apply the study drug specific dose-modification guidelines accordingly. If the toxicity is not clearly attributable to either individual study drug, then the causality should be attributed to both study drugs. Any time that causality is attributed to both study drugs, AE management should occur per investigator discretion and, if needed, in consultation with the sponsor. Investigators are encouraged to consult the sponsor for additional guidance.

Dosing interruptions of any study drug are permitted for reasons not related to study treatment (for example, minor surgery, unrelated medical events, patient vacation, and/or holidays). The reason for interruption should be documented on the CRF. Efficacy Evaluations.

### Inclusion Criteria

[1] Patients are eligible to be included in the part of the study for Compound A in combination with nab-paclitaxel/gemcitabine study only if they meet all of the following criteria: Metastatic PDAC.
[2] Have at least 1 measurable lesion assessable using standard techniques by RECIST 1.1 for patients in Part D (Eisenhauer et al. 2009). Positron emission tomography (PET) scans and ultrasounds may not be used for diagnostic purposes.
[3] must be able and willing to undergo mandatory tumor biopsies which will be collected, following determination of eligibility, before treatment (≤28 days before C1D1) and after 2 weeks of treatment (in C1 during Days 16-20.). Archived tissue obtained from a recent biopsy may be permitted for the pretreatment sample, after discussion between Lilly CRP/CRS and investigator, if a patient has not received any therapies for the disease between the time biopsy was obtained to the start of Compound A treatment. The decision to use an archived tissue sample will be documented in writing.
[4] have a performance status (PS) of 0 to 1 on the Eastern Cooperative Oncology (Group (ECOG) scale (Oken et al. 1982)
[5] have discontinued previous treatments for cancer and have resolution, except where otherwise stated in the inclusion criteria, of all clinically significant toxic effects of prior chemotherapy, surgery, or radiotherapy to Grade ≤1 by National Cancer Institute (NCI) Common Terminology Criteria for Adverse Events (CTCAE), Version 4.0 (v 4.0) (CTEP 2009).
[6] at the discretion of the investigator, patients with hormone-refractory prostate cancer who are stable on gonadotropin-releasing hormone (GnRH) agonist therapy/patients with breast cancer who are stable on anti-estrogen therapy (for example, an aromatase inhibitor) may continue that treatment while enrolled in this study.
[7] have adequate organ function, as defined below:

### System Laboratory Value

### Hematologic

### ANC 1.5×109/L

### Platelets 100×109/L

Hemoglobin ≥8 g/dL
Transfusions to increase the patient's hemoglobin level to 8 g/dL are not permitted within 1 week prior to the baseline hematology profile

### Hepatic

Total bilirubin ≤1.5×ULN

### ALT and AST ≤2.5×ULN OR

≤5×ULN if the liver has tumor involvement

### Renal

Serum creatinine OR
Calculated creatinine clearance
≤1.5×ULN OR260 mL/min
Abbreviations: ALT=alanine aminotransferase; AST=aspartate aminotransferase; ANC=absolute neutrophil count; ULN=upper limit of normal.
[8] are at least 18 years old at the time of screening.
[9] are male patients who are sterile (including vasectomy confirmed by post vasectomy semen analysis) or agree to use an effective method of contraception and not to donate sperm or practice total abstinence from heterosexual activity, starting with the first dose of study treatment, during the study, and for at least 6 months following the last dose of study treatment, or longer, if determined by country requirements.
[10] are female patients of non-childbearing potential (defined below), or are female patients of child-bearing potential who are not pregnant, as confirmed by a serum pregnancy test within 7 days prior to receiving first dose of study treatment and who agree to use 2 methods of birth control (hormonal or intrauterine plus a barrier method) or practice total abstinence from heterosexual activity during the study for at least 6 months following the last dose of the study treatment, or longer, if determined by country requirement
[11] have given written informed consent/assent prior to any study-specific procedures
[12] are able to swallow capsules or tablets
[13] have an estimated life expectancy of 212 weeks, in the judgment of the investigator.

### Exclusion Criteria

Patients will be excluded from the study if they meet any of the following criteria:

[14] have a serious concomitant systemic disorder (for example, active infection or a gastrointestinal disorder causing clinically significant symptoms such as nausea, vomiting or diarrhea, or profound immune suppression) that, in the opinion of the investigator, would compromise the patient's ability to adhere to the protocol.
[15] have a known human immunodeficiency virus (HIV) infection or known activated/reactivated hepatitis A, B, or C (screening is not required).
[16] have symptomatic central nervous system (CNS) malignancy or metastasis (screening not required) Patients with treated CNS metastases are eligible for this study if they are not currently receiving corticosteroids for their CNS metastasis and/or anticonvulsants, and their disease is asymptomatic and radiographically stable for at least 60 days.
[17] have current hematologic malignancies, acute or chronic leukemia
[18] have a second primary malignancy that in the judgment of the investigator or Lilly may affect the interpretation of results
[19] have prior malignancies. Patients with carcinoma in situ of any origin and patients with prior malignancies who are in remission and whose likelihood of recurrence is very low, as judged by the Lilly CRP, are eligible for this study. The Lilly CRP will approve enrollment of patients with prior malignancies in remission before these patients are enrolled.
[20] Have a mean QT interval corrected for heart rate (QTc) of 2470 msec on screening electrocardiogram (ECG) as calculated using the Bazett's formula at several consecutive days of assessment.
[21] are currently enrolled in a clinical trial involving an investigational product or any other type of medical research judged not to be scientifically or medically compatible with this study
[22] have participated, within the last 28 days in a clinical trial involving an investigational product.
[23] have previously completed or withdrawn from this study or any other study investigating an ERK1/2 inhibitor.
[24] if female, is pregnant, breastfeeding, or planning to become pregnant.
[25] have history or findings of central or branch retinal artery or venous occlusion with significant vision loss or other retinal diseases that cause current visual impairment or would likely cause visual impairment over the time period of the study, as assessed by an ophthalmologist
[26] currently using concomitant medications that are strong inhibitors or inducers of CYP3A4.
Discontinuation from Study Treatment
Patients receiving Compound A with one of the combination partners are considered to have discontinued from study treatment when all study treatments are no longer administered. Patients will be discontinued from study treatment in the following circumstances:
the patient is enrolled in any other clinical trial involving an investigational product or any other type of medical research judged not to be scientifically or medically compatible with this study
the patient becomes pregnant during the study
the patient is significantly noncompliant with study procedures and/or treatment
disease progression
unacceptable toxicity
the patient has had 2 dose reductions and experiences an AE that would cause a third dose reduction
the patient, for any reason, requires treatment with another therapeutic agent that has been demonstrated to be effective for treatment of the study indication. Discontinuation from study treatment will occur prior to introduction of the new agent
the investigator decides that the patient should be discontinued from study treatment
the patient requests to be discontinued from study treatment
the patient's designee (for example, parents, legal guardian, or caregiver) requests that the patient be discontinued from study treatment Patients who are discontinued from study treatment will have follow-up procedures performed.

### Study Endpoints and Efficacy Assessments

Palpable or visible tumors will be measured on day 1 of each cycle. Hematology studies and clinical chemistry studies will be performed on days 1, 8 and 15 of each cycle. Urinalysis will be performed on day 1 of each cycle beginning with cycle 2.

Computed tomography (CT) scans, including spiral CT, are the preferred methods of measurement (CT scan thickness recommended to be ≤5 mm); however, magnetic resonance imaging (MRI) is also acceptable in certain situations, such as when body scans are indicated or if there is a concern about radiation exposure associated with CT. Intravenous and oral contrast is required unless medically contraindicated.

The CT portion of a positron emission tomography (PET)-CT scan may be used as method of response assessment if the site can document that the CT is of identical diagnostic quality to a diagnostic CT (with intravenous and oral contrast). A PET scan alone or as part of a PET-CT may be performed for additional analyses but cannot be used to assess response according to RECIST v. 1.1 (Eisenhauer et al. 2009). The method of tumor assessment used at baseline must be used consistently throughout the study. Each patient's full extent of disease will be assessed using RECIST v1.1 (Eisenhauer et al. 2009).

1. A method of treating pancreatic cancer in a patient, comprising administering to the patient an effective amount of 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and gemcitabine, or a pharmaceutically acceptable salt thereof.

2. The method of claim 1 further comprising nab-paclitaxel.

3. The method of claim 1, wherein the pancreatic cancer is PDAC.

4. A kit for pancreatic cancer comprising an oral agent including an effective component 6,6-dimethyl-2-{2-[(1-methyl-1H-pyrazol-5-yl)amino]pyrimidin-4-yl}-5-[2-(morpholin-4-yl)ethyl]-5,6-dihydro-4H-thieno[2,3-c]pyrrol-4-one, or a pharmaceutically acceptable salt thereof, and an injection agent including an effective component gemcitabine, or a pharmaceutically acceptable salt thereof.

5. The kit of claim 4 further comprising an injection agent including an effective component nab-paclitaxel.

6. The kit of claim 4, wherein the pancreatic cancer is PDAC.

7. The kit of claim 4, further comprising one or more pharmaceutically acceptable carriers, diluents, or excipients.

8-12. (canceled)

13. The method of claim 2, wherein the pancreatic cancer is PDAC.

14. The kit of claim 5, wherein the pancreatic cancer is PDAC.

15. The kit of claim 5, further comprising one or more pharmaceutically acceptable carriers, diluents, or excipients.

16. The kit of claim 6, further comprising one or more pharmaceutically acceptable carriers, diluents, or excipients.
